SEQUENCE LISTING (9 sequences)

SEQ ID NO: 1 - protein, 584 aa, Saccharomyces cerevisiae
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 2 - protein, 312 aa, Saccharomyces cerevisiae
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFK

SEQ ID NO: 3 - protein, 21 aa, Artificial Sequence
  Synthetic construct
GAGPARPAGLPPATYYDSLAV

SEQ ID NO: 4 - protein, 6 aa, Artificial Sequence
  Synthetic construct
AGGGGV

SEQ ID NO: 5 - protein, 917 aa, Artificial Sequence
  Synthetic construct
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSALGAGPARPAGLPPATYYDSLAVMPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFK

SEQ ID NO: 6 - protein, 902 aa, Artificial Sequence
  Synthetic construct
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSALAGGGGVMPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFK

SEQ ID NO: 7 - protein, 917 aa, Artificial Sequence
  Synthetic construct
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFKGAGPARPAGLPPATYYDSLAVMSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 8 - protein, 902 aa, Artificial Sequence
  Synthetic construct
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFKAGGGGVMSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 9 - protein, 1173 aa, Artificial Sequence
  Synthetic construct
MTSPVIGTPWKKLNAPVSEAAIEGVDKYWRVANYLSIGQIYLRSNPLMKEPFTREDVKHRLVGHWGTTPGLNFLIGHINRFIAEHQQNTVIIMGPGHGGPAGTAQSYLDGTYTEYYPKITKDEAGLQKFFRQFSYPGGIPSHFAPETPGSIHEGGELGYALSHAYGAVMNNPSLFVPAIVGDGEAETGPLATGWQSNKLVNPRTDGIVLPILHLNGYKIANPTILSRISDEELHEFFHGMGYEPYEFVAGFDDEDHMSIHRRFADMFETIFDEICDIKAEAQTNDVTRPFYPMIIFRTPKGWTCPKFIDGKKTEGSWRAHQVPLASARDTEAHFEVLKNWLKSYKPEELFNEDGSIKEDVLSFMPQGELRIGQNPNANGGRIREDLKLPNLDDYEVKEVKEFGHGWGQLEATRRLGVYTRDVIKNNPDSFRIFGPDETASNRLQAAYEVTNKQWDAGYLSELVDEHMAVTGQVTEQLSEHQMEGFLEAYLLTGRHGIWSSYESFVHVIDSMLNQHAKWLEATVREIPWRKPISSMNLLVSSHVWRQDHNGFSHQDPGVTSVLLNKTFNNDHVIGIYFPVDSNMLLAVGEKVYKSTNMINAIFAGKQPAATWLTLDEAREELEKGAAEWKWASNAKNNDEVQVVLAGIGDVPQQELMAAADKLNKLGVKFKVVNIVDLLKLQSAKENNEALTDEEFTELFTADKPVLLAYHSYAHDVRGLIFDRPNHDNFNVHGYKEQGSTTTPYDMVRVNDMDRYELTAEALRMVDADKYADEIKKLEDFRLEAFQFAVDKGYDHPDYTDWVWPGVKTDKPGAVTATAATAGDNEGAGPARPAGLPPATYYDSLAVTSMDLFESLAQKITGKDQTIVFPEGTEPRIVGAAARLAADGLVKPIVLGATDKVQAVANDLNADLTGVQVLDPATYPAEDKQAMLDALVERRKGKNTPEQAAKMLEDENYFGTMLVYMGKADGMVSGAIHPTGDTVRPALQIIKTKPGSHRISGAFIMQKGEERYVFADCAINIDPDADTLAEIATQSAATAKVFDIDPKVAMLSFSTKGSAKGEMVTKVQEATAKAQAAEPELAIDGELQFDAAFVEKVGLQKAPGSKVAGHANVFVFPELQSGNIGYKIAQRFGHFEAVGPVLQGLNKPVSDLSRGCSEEDVYKVAIITAAQGLA

(19) United States
(12) Patent Application Publication
(10) Pub. No.: US 2021/0047660 A1
(43) Pub. Date: Feb. 18, 2021

(54) COMPOSITIONS AND METHODS FOR INCREASING ETHANOL PRODUCTION BY YEAST USING GCY1 AND DAK1

(71) Applicant: DANISCO US INC., Palo Alto, CA (US)

(72) Inventors: Quin Qun Zhu, West Chester, PA (US); Paula Johanna Maria Teunissen, Palo Alto, CA (US)

(21) Appl. No.: 16/976,525

(22) PCT Filed: Mar. 4, 2019
(86) PCT No.: PCT/US2019/020551

§ 371 Date: Aug. 28, 2020

Related U.S. Application Data
(60) Provisional application No. 62/639,161, filed on Mar. 6, 2018.

(51) Int. Cl. C12P 7/06; C12N 9/04; C12N 9/12

(52) U.S. Cl. CPC C12P 7/06; C12Y 101/01006; C12N 9/12; C12N 9/0006

(57) ABSTRACT

Described are compositions and methods relating to yeast expressing glycerol dehydrogenase and dihydroxyacetone kinase polypeptides in combination with an exogenous phosphoketolase pathway, as well as to bifunctional glycerol dehydrogenase-dihydroxyacetone kinase fusion polypeptides, and their various and combined uses in starch hydrolysis processes for alcohol production.

### TECHNICAL FIELD

The present compositions and methods relate to yeast expressing glycerol dehydrogenase and dihydroxyacetone kinase polypeptides in combination with an exogenous phosphoketolase pathway, as well as to bifunctional glycerol dehydrogenase-dihydroxyacetone kinase fusion polypeptides, and their use thereof in starch hydrolysis processes for alcohol production.

### BACKGROUND

Yeast-based ethanol production is based on the conversion of sugars into ethanol. The current annual fuel ethanol production by this method is about 90 billion liters worldwide. It is estimated that about 70% of the cost of ethanol production is the feedstock. Since the ethanol production volume is so large, even small yield improvements have massive economic impact for the industry. The conversion of one mole of glucose into two moles of ethanol and two moles of carbon dioxide is redox-neutral, with the maximum theoretical yield being about 51%. The current industrial yield is about 45%; therefore, there are opportunities to increase ethanol production.

Carbon dioxide, glycerol and yeast biomass are the major by-products of ethanol fermentation. During yeast growth and fermentation, a surplus of NADH is generated, which is used to produce glycerol for the purposes of redox balance and osmotic protection. Glycerol is considered a low value product and several approaches have been taken to reduce glycerol production. However, reducing glycerol synthesis can result in the increase of other metabolic by-products, such as acetate. Acetate is not a desirable by-product, as it adversely affects the alcohol production rate, titer and yield of yeast fermentation.

Engineered yeast cells having a heterologous phosphoketolase pathway have been previously described (e.g., WO2015148272). These cells express heterologous phosphoketolase (PKL; EC 4.1.2.9) and phosphotransacetylase (PTA; EC 2.3.1.8), optionally with other enzymes, to channel carbon flux away from the glycerol pathway and toward the synthesis of acetyl-coA, which is then converted to ethanol. These cells are capable of increased ethanol production in a fermentation process when compared to otherwise-identical parent yeast cells.

Despite advances in yeast productivity, the need exists to further modify yeast metabolic pathways to maximize ethanol production, while not increasing the production of undesirable by-products.

### SUMMARY

The present compositions and methods relate to yeast cells expressing glycerol dehydrogenase and dihydroxyacetone kinase polypeptides in combination with an exogenous phosphoketolase pathway, bifunctional glycerol dehydrogenase-dihydroxyacetone kinase fusion polypeptides and yeast cells expressing them, and their various and combined uses in starch hydrolysis processes for alcohol production. Aspects and embodiments of the compositions and methods are described in the following, independently-numbered paragraphs.

1. In one aspect, a fusion polypeptide is provided comprising a first amino acid sequence having glycerol dehydrogenase activity fused to a second amino acid sequence having dihydroxyacetone kinase activity, wherein the fusion polypeptide, when expressed in a yeast cell, is capable of converting glycerol to dihydroxyacetone phosphate.

2. In some embodiments of the fusion polypeptide of paragraph 1, the first amino acid sequence and second amino acid sequence are fused via a linker peptide.

3. In some embodiments of the fusion polypeptide of paragraph 1 or 2, the first amino acid sequence is present at the N-terminus of the fusion polypeptide and second amino acid sequence is present at the C-terminus of the fusion polypeptide.

4. In some embodiments of the fusion polypeptide of paragraph 1 or 2, the second amino acid sequence is present at the N-terminus of the fusion polypeptide and first amino acid sequence is present at the C-terminus of the fusion polypeptide.

5. In some embodiments of the fusion polypeptide of any of the preceding paragraphs, the first amino acid sequence is the glycerol dehydrogenase from a Saccharomyces sp. or a structural or functional homolog, thereof.

6. In some embodiments of the fusion polypeptide of any of the preceding paragraphs, the second amino acid sequence is the dihydroxyacetone kinase from a Saccharomyces sp. or a structural or functional homolog, thereof.

7. In another aspect, a DNA sequence encoding the fusion polypeptide of any of the preceding paragraphs is provided, optionally capable of overexpressing the fusion polypeptide compared to the individual expression levels of either or both GCY1 or DAK1, based on mRNA levels, compared to a parental yeast not harboring the DNA sequence.

8. In another aspect, yeast cells comprising the DNA sequence of paragraph 7 are provided.

9. In another aspect, yeast cells expressing or overexpressing the fusion polypeptide of any of paragraphs 1-8 are provided.

10. In some embodiments of the yeast cells of paragraph 9, the yeast cells further comprise an exogenous phosphoketolase pathway.

11. In some embodiments of the yeast cells of any of paragraphs 9 or 10, the yeast cells further comprise an alteration in the glycerol pathway and/or the acetyl-CoA pathway.

12. In some embodiments of the yeast cells of any of paragraphs 9-11, the yeast cells do not additionally overexpress separate polypeptides having glycerol dehydrogenase activity and/or dihydroxyacetone kinase activity.

13. In another aspect, modified yeast cells are provided, comprising a genetic modification that causes the cells to overexpress a polypeptide having glycerol dehydrogenase activity and overexpress a polypeptide having dihydroxyacetone kinase activity, and which modified yeast cells further comprise an exogenous phosphoketolase pathway.

14. In some embodiments of the modified yeast cells of paragraph 13, the polypeptide having glycerol dehydrogenase activity and the polypeptide having dihydroxyacetone kinase activity are produced as a bifunctional fusion polypeptide capable of converting glycerol to dihydroxyacetone phosphate.

15. In some embodiments of the modified yeast cells of paragraph 13 or 14, the yeast produce a reduced amount of dihydroxyacetone compared to otherwise unmodified or parental yeast.

16. In some embodiments the modified yeast cells of any of paragraphs 13-15 further comprise an alteration in the glycerol pathway and/or the acetyl-CoA pathway

17. In some embodiments the modified yeast of any of paragraphs 13-16, further comprise an exogenous gene encoding a carbohydrate processing enzyme.

18. In some embodiments of the modified yeast cells of any of paragraphs 13-17, the yeast cells are from a Saccharomyces spp.

19. In another aspect, a method for increasing alcohol production by yeast cells during fermentation of a carbohydrate substrate is provided, comprising contacting the carbohydrate substrate with modified yeast cells having an exogenous phosphoketolase pathway and producing a polypeptide having glycerol dehydrogenase activity and a polypeptide having dihydroxyacetone kinase activity, wherein the modified yeast cells produce during fermentation an increased amount of ethanol compared to yeast cells that do not produce the polypeptide having glycerol dehydrogenase activity and/or the polypeptide having dihydroxyacetone kinase activity.

20. In some embodiments of the method of paragraph 19, the modified yeast cells are the yeast cells of any of paragraphs 9-11.

21. In some embodiments of the method of any paragraph 19 or 20, the polypeptide having glycerol dehydrogenase activity and the polypeptide having dihydroxyacetone kinase activity are expressed as a bifunctional fusion polypeptide capable of converting glycerol to dihydroxyacetone phosphate.

22. In some embodiments of the method of paragraph 21, the bifunctional fusion polypeptide is the fusion polypeptide of any of paragraphs 1-6.

These and other aspects and embodiments of present modified cells and methods will be apparent from the description, including the accompanying Figures.

### BRIEF DESCRIPTION OF THE DRAWINGS

FIG. 1 is a map of the 2,018-bp ura3-loxP-KanMX-loxP-ura3 cassette released from plasmid pTOPO II-Blunt ura3-loxP-KanMX-loxP-ura3 by digestion with EcoRI.

FIG. 2 is a map of the 4,776-bp GCY1-L1-DAK1 expression cassette released from plasmid pZKIIC-YL1K by digestion with SwaI.

FIG. 3 is a map of the 4,731-bp GCY1-L2-DAK1 expression cassette released from plasmid pZKIIC-YL2K by digestion with SwaI.

FIG. 4 is a map of the 4,776-bp DAK1-L1-GCY1 expression cassette released from plasmid pZKIIC-KL1Y by digestion with SwaI.

FIG. 5 is a map of the 4,731-bp DAK1-L2-GCY1 expression cassette released from plasmid pZKIIC-KL2Y by digestion with SwaI.

FIG. 6 is a map of the 5,668-bp GCY1 and DAK1 separate expression cassettes released from plasmid pZKIIC-HYKK by digestion with SwaI.

FIG. 7 is a map of the 12,372-bp PKL pathway expression cassettes released from plasmid pZK41W-GLAF12 by digestion with SwaI.

### DETAILED DESCRIPTION

### I. Definitions

Prior to describing the present yeast strains and methods in detail, the following terms are defined for clarity. Terms not defined should be accorded their ordinary meanings as used in the relevant art.

As used herein, “alcohol” refer to an organic compound in which a hydroxyl functional group (—OH) is bound to a saturated carbon atom.

As used herein, “yeast cells,” “yeast strains” or simply “yeast” refer to organisms from the phyla Ascomycota and Basidiomycota. An exemplary yeast is budding yeast from the order Saccharomycetales. A particular example of yeast is Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “engineered yeast cells,” “variant yeast cells,” “modified yeast cells,” or similar phrases, refer to yeast that include genetic modifications and characteristics described herein. Variant/modified yeast do not include naturally occurring yeast.

As used herein, the term “exogenous” refers to a gene, or its encoded protein, activity or affect, that is not found in a subject organism in nature and is introduced to the organism by genetic manipulation.

As used herein, the term “phosphoketolase pathway” refers to a cellular metabolic pathway that includes a phosphoketolase (PKL) enzyme. Additional enzymes that may be included in a PKL pathway include, but are not limited to, phosphotransacetylase (PTA), acetylating acetyl dehydrogenase (AADH) and/or acetyl-CoA synthase (ACS).

As used herein, the terms “polypeptide,” “protein,” “amino acid sequences” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or different classes of organisms (e.g., bacteria and fungi), or artificially designed. Related proteins also encompass homologs determined by primary sequence analysis, determined by secondary or tertiary structure analysis, or determined by enzyme activities, or determined by immunological cross-reactivity.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′S, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the term “expressing a polypeptide” and similar terms refers to the cellular process of producing a polypeptide using the translation machinery (e.g., ribosomes) of the cell. In this context, “expressing” and “producing” are used without distinction.

As used herein, “overexpressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a nucleic acid that includes an amino acid coding sequence, promoters, terminators, and other nucleic acid sequence needed to allow the encoded polypeptide to be produced in a cell. Expression cassettes can be exogenous (i.e., introduced into a cell) or endogenous (i.e., extant in a cell).

As used herein, the terms “wild-type” and “native” are used interchangeably and refer to genes proteins or strains found in nature.

As used herein, the terms “fused” and “fusion” with respect to two polypeptides refer to a physical linkage causing the polypeptide to become a single molecule.

As used herein, the terms “wild-type” and “native” are used interchangeably and refer to genes, proteins or strains found in nature.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed at high levels. The protein of interest is encoded by an endogenous gene, a modified endogenous gene or a heterologous gene (i.e., gene of interest”) relative to the parental strain. The protein of interest can be expressed intracellularly or as a secreted protein.

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements. Deletion of a gene also refers to the deletion a part of the coding sequence, or a part of promoter immediately or not immediately adjacent to the coding sequence, where there is no functional activity of the interested gene existed in the engineered cell.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a signal transducer, a receptor, a transporter, a transcription factor, a translation factor, a co-factor, a binding activity, a surface-active property, or the like, and which has not been mutagenized, truncated, or otherwise modified to abolish or reduce that activity. Functional polypeptides can be thermostable or thermolabile, as specified.

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:
EC enzyme commission
GCY glycerol dehydrogenase
DAK dihydroxyacetone kinase
PKL phosphoketolase
PTA phosphotransacetylase
XFP xylulose 5-phosphate/fructose 6-phosphate phosphoketolase AADH acetaldehyde dehydrogenases
ADH alcohol dehydrogenase
EtOH ethanol
AA α-amylase
GA glucoamylase
TrGA Trichoderma glucoamylase
° C. degrees Centigrade
bp base pairs
DHA dihydroxyacetone
DNA deoxyribonucleic acid
DP degree of polymerization
ds or DS dry solids
g or gm gram
g/L grams per liter
GAU/g ds glucoamylase units per gram dry solids
H₂O water
HPLC high performance liquid chromatography
hr or h hour
kg kilogram
M molar
mg milligram
mL or ml milliliter
ml/min milliliter per minute
mM millimolar
N normal
nm nanometer
OD optical density
PCR polymerase chain reaction
ppm parts per million
SSCU/g ds fungal alpha-amylase units per gram dry solids
Δ relating to a deletion
μg microgram
μL and μl microliter
micromolar
SSF simultaneous saccharification and fementation
MTP microtiter plate

### II. Introduction

Over-expression of two genes together, glycerol dehydrogenase (GCY1) and dihydroxyacetone kinase (DAK1), have been demonstrated to reduce glycerol production and increase ethanol yield in wild-type yeast strains (Zhang et al. (2013) J. Ind. Microbiol. Biotechnol. 40:1153-60). No protein has heretofore been identified that has both glycerol dehydrogenase and dihydroxyacetone kinase activities.

A first aspect of the present compositions and methods relates to a bifunctional GCY1-DAK1 fusion polypeptide, which includes active portions of both enzymes. The fusion polypeptide is at least as efficient, and in some cases significantly more efficient, for enhancing ethanol production in yeast and reduces the accumulation of the toxic intermediate, dihydroxyacetone (DHA) in the yeast.

A second aspect of the present compositions and methods relates to the overexpression of GCY1 and DAK1, separately or together as a bifunctional fusion polypeptide, in yeast harboring a PKL pathway to further increase the production of ethanol compared to yeast harboring the PKL pathway alone. Such engineered yeast is ideal for use in the fuel ethanol industry based on its ability to produce significantly more ethanol than parental yeast while producing substantially less glycerol as wasted carbon.

A third aspect of the present compositions and methods relates specifically to the expression of GCY1 and DAK1 together as a bifunctional fusion polypeptide, as opposed to separately, in yeast harboring a PKL pathway to further increase the production of ethanol compared to yeast harboring the PKL pathway alone. Such highly-engineered yeast is even more ideal for use in the fuel ethanol industry based on its ability to produce more ethanol, less glycerol, and less DHA.

### III. GCY1 and DAK1 Polypeptides

The exemplified GCY1 and DAK1 polypeptides are the full-length GCY1 from Saccharomyces cerevisiae S288C (GenBank Accession No. NP_014763) and the full-length DAK1 from S. cerevisiae S288C (GenBank Accession No. NP_013641).

The amino acid sequence of DAK1 is shown, below (SEQ ID NO: 1):

MSAKSFEVTDPVNSSLKGFALANPSITLVPEEKILFRKTDSDKIALISGG
GSGHEPTHAGFIGKGMLSGAVVGEIFASPSTKQILNAIRLVNENASGVLL
IVKNYTGDVLHFGLSAERARALGINCRVAVIGDDVAVGREKGGMVGRRAL
AGTVLVHKIVGAFAEEYSSKYGLDGTAKVAKIINDNLVTIGSSLDHCKVP
GRKFESELNEKQMELGMGIHNEPGVKVLDPIPSTEDLISKYMLPKLLDPN
DKDRAFVKFDEDDEVVLLVNNLGGVSNFVISSITSKTTDFLKENYNITPV
QTIAGTLMTSFNGNGFSITLLNATKATKALQSDFEEIKSVLDLLNAFTNA
PGWPIADFEKTSAPSVNDDLLHNEVTAKAVGTYDFDKFAEWMKSGAEQVI
KSEPHITELDNQVGDGDCGYTLVAGVKGITENLDKLSKDSLSQAVAQISD
FIEGSMGGTSGGLYSILLSGFSHGLIQVCKSKDEPVTKEIVAKSLGIALD
TLYKYTKARKGSSTMIDALEPFVKEFTASKDFNKAVKAAEEGAKSTATFE
AKFGRASYVGDSSQVEDPGAVGLCEFLKGVQSAL

The amino acid sequence of GCY1 is shown, below (SEQ ID NO: 2):

MPATLHDSTKILSLNTGAQIPQIGLGTWQSKENDAYKAVLTALKDGYRHI
DTAAIYRNEDQVGQAIKDSGVPREEIFVTTKLWCTQHHEPEVALDQSLKR
LGLDYVDLYLMHWPARLDPAYIKNEDILSVPTKKDGSRAVDITNWNFIKT
WELMQELPKTGKTKAVGVSNFSINNLKDLLASQGNKLTPAANQVEIHPLL
PQDELINFCKSKGIVVEAYSPLGSTDAPLLKEPVILEIAKKNNVQPGHVV
ISWHVQRGYVVLPKSVNPDRIKTNRKIFTLSTEDFEAINNISKEKGEKRV
VHPNWSPFEVFK

GCY1 and DAK1 enzymes are well-known enzymes and a large number have been cloned and characterized. Public databases include many GCY1 and DAK1 sequences. Other GCY1 and DAK1 polypeptides are expected to work as described herein, including structural and functional homologs, including, but not limited to, those having at least 70%, at least 80%, at least 85%, at least 90%, at least 95%, or more amino acid sequence identity and/or homology to the exemplified GCY1 and DAK1.

In some embodiments, GCY1 and DAK1 polypeptides include substitutions that do not substantially affect the structure and/or function of the polypeptide. Exemplary substitutions are conservative mutations, as summarized in Table 1.

TABLE 1
Exemplary amino acid substitutions

| Original Amino | | |
|---|---|---|
| Acid Residue | Code | Acceptable Substitutions |
| Alanine | A | D-Ala, Gly, β-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4- carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

In some embodiments, yeast expressing (i.e., producing) GCY1 and DAK1 polypeptides produce at least 0.5%, at least 0.8%, at least 1%, at least 2%, at least 3%, at least 4%, at least 5%, or even at least 6% or more ethanol from a carbohydrate substrate than yeast not expressing the polypeptides. In some embodiments, the yeast expressing GCY1 and DAK1 polypeptides produce at least 3%, at least 5%, at least 10%, at least 15%, or even at least 20%, less glycerol from a carbohydrate substrate than yeast not expressing the polypeptides.

In some embodiments, the present yeast overexpress GCY1 and DAK1 polypeptides by at least 50%, at least 100%, at least 200%, at least 300%, at least 500%, or more, and/or at least 2-fold, at least 5-fold, at least 10-fold, at least 20-fold, or more compared to the normal individual expression levels of GCY1 and DAK1, based on mRNA levels.

### IV. Bifunctional GCY1-DAK1 Fusion Polypeptides

The exemplified bifunctional GCY1-DAK1 fusion polypeptides are relatively simple in design in that they include the full-length GCY1 corresponding to SEQ ID NO: 2 and full-length DAK1 corresponding to SEQ ID NO: 1 connected via one of two different linkers (i.e., L1 or L2), in two different orders of arrangement.

The amino acid sequence of Linker 1 (L1) is shown, below (SEQ ID NO: 3):

GAGPARPAGLPPATYYDSLAV

The amino acid sequence of Linker 2 (L2) is shown, below (SEQ ID NO: 4):

AGGGGV

The amino acid sequence of DAK1-L1-GCY1 is shown, below (SEQ ID NO: 5), with the linker in bold and italic:

MSAKSFEVTDPVNSSLKGFALANPSITLVPEEKILFRKTDSDKIALIS
GGGSGHEPTHAGFIGKGMLSGAVVGEIFASPSTKQILNAIRLVNENAS
GVLLIVKNYTGDVLHFGLSAERARALGINCRVAVIGDDVAVGREKGGM
VGRRALAGTVLVHKIVGAFAEEYSSKYGLDGTAKVAKIINDNLVTIGS
SLDHCKVPGRKFESELNEKQMELGMGIHNEPGVKVLDPIPSTEDLISK
YMLPKLLDPNDKDRAFVKFDEDDEVVLLVNNLGGVSNFVISSITSKTT
DFLKENYNITPVQTIAGTLMTSFNGNGFSITLLNATKATKALQSDFEE
IKSVLDLLNAFTNAPGWPIADFEKTSAPSVNDDLLHNEVTAKAVGTYD
FDKFAEWMKSGAEQVIKSEPHITELDNQVGDGDCGYTLVAGVKGITEN
LDKLSKDSLSQAVAQISDFIEGSMGGTSGGLYSILLSGFSHGLIQVCK
SKDEPVTKEIVAKSLGIALDTLYKYTKARKGSSTMIDALEPFVKEFTA
SKDFNKAVKAAEEGAKSTATFEAKFGRASYVGDSSQVEDPGAVGLCEF
LKGVQSAL [symbol] MPATLHDSTKILSLNTGAQ
IPQIGLGTWQSKENDAYKAVLTALKDGYRHIDTAAIYRNEDQVGQAIK
DSGVPREEIFVTTKLWCTQHHEPEVALDQSLKRLGLDYVDLYLMHWPA
RLDPAYIKNEDILSVPTKKDGSRAVDITNWNFIKTWELMQELPKTGKT
KAVGVSNFSINNLKDLLASQGNKLTPAANQVEIHPLLPQDELINFCKS
KGIVVEAYSPLGSTDAPLLKEPVILEIAKKNNVQPGHVVISWHVQRGY
VVLPKSVNPDRIKTNRKIFTLSTEDFEAINNISKEKGEKRVVHPNWSP
FEVFK

The amino acid sequence of DAK1-L2-GCY1 is shown, below (SEQ ID NO: 6) with the linker in bold and italic:

MSAKSFEVTDPVNSSLKGFALANPSITLVPEEKILFRKTDSDKIALISGG
GSGHEPTHAGFIGKGMLSGAVVGEIFASPSTKQILNAIRLVNENASGVLL
IVKNYTGDVLHFGLSAERARALGINCRVAVIGDDVAVGREKGGMVGRRAL
AGTVLVHKIVGAFAEEYSSKYGLDGTAKVAKIINDNLVTIGSSLDHCKVP
GRKFESELNEKQMELGMGIHNEPGVKVLDPIPSTEDLISKYMLPKLLDPN
DKDRAFVKFDEDDEVVLLVNNLGGVSNFVISSITSKTTDFLKENYNITPV
QTIAGTLMTSFNGNGFSITLLNATKATKALQSDFEEIKSVLDLLNAFTNA
PGWPIADFEKTSAPSVNDDLLHNEVTAKAVGTYDFDKFAEWMKSGAEQVI
KSEPHITELDNQVGDGDCGYTLVAGVKGITENLDKLSKDSLSQAVAQISD
FIEGSMGGTSGGLYSILLSGFSHGLIQVCKSKDEPVTKEIVAKSLGIALD
TLYKYTKARKGSSTMIDALEPFVKEFTASKDFNKAVKAAEEGAKSTATFE
AKFGRASYVGDSSQVEDPGAVGLCEFLKGVQSAL [symbol] MPATLHDSTK
ILSLNTGAQIPQIGLGTWQSKENDAYKAVLTALKDGYRHIDTAAIYRNED
QVGQAIKDSGVPREEIFVTTKLWCTQHHEPEVALDQSLKRLGLDYVDLYL
MHWPARLDPAYIKNEDILSVPTKKDGSRAVDITNWNFIKTWELMQELPKT
GKTKAVGVSNFSINNLKDLLASQGNKLTPAANQVEIHPLLPQDELINFCK
SKGIVVEAYSPLGSTDAPLLKEPVILEIAKKNNVQPGHVVISWHVQRGYV
VLPKSVNPDRIKTNRKIFTLSTEDFEAINNISKEKGEKRVVHPNWSPFEV
FK

The amino acid sequence of GCY1-L1-DAK1 is shown, below (SEQ ID NO: 7), with the linker in bold and italic:

MPATLHDSTKILSLNTGAQIPQIGLGTWQSKENDAYKAVLTALKDGYRHI
DTAAIYRNEDQVGQAIKDSGVPREEIFVTTKLWCTQHHEPEVALDQSLKR
LGLDYVDLYLMHWPARLDPAYIKNEDILSVPTKKDGSRAVDITNWNFIKT
WELMQELPKTGKTKAVGVSNFSINNLKDLLASQGNKLTPAANQVEIHPLL
PQDELINFCKSKGIVVEAYSPLGSTDAPLLKEPVILEIAKKNNVQPGHVV
ISWHVQRGYVVLPKSVNPDRIKTNRKIFTLSTEDFEAINNISKEKGEKRV
VHPNWSPFEVFK [symbol] MSAKSFEVTDPVNSSLK
GFALANPSITLVPEEKILFRKTDSDKIALISGGGSGHEPTHAGFIGKGML
SGAVVGEIFASPSTKQILNAIRLVNENASGVLLIVKNYTGDVLHFGLSAE
RARALGINCRVAVIGDDVAVGREKGGMVGRRALAGTVLVHKIVGAFAEEY
SSKYGLDGTAKVAKIINDNLVTIGSSLDHCKVPGRKFESELNEKQMELGM
GIHNEPGVKVLDPIPSTEDLISKYMLPKLLDPNDKDRAFVKFDEDDEVVL
LVNNLGGVSNFVISSITSKTTDFLKENYNITPVQTIAGTLMTSFNGNGFS
ITLLNATKATKALQSDFEEIKSVLDLLNAFTNAPGWPIADFEKTSAPSVN
DDLLHNEVTAKAVGTYDFDKFAEWMKSGAEQVIKSEPHITELDNQVGDGD
CGYTLVAGVKGITENLDKLSKDSLSQAVAQISDFIEGSMGGTSGGLYSIL
LSGFSHGLIQVCKSKDEPVTKEIVAKSLGIALDTLYKYTKARKGSSTMID
ALEPFVKEFTASKDFNKAVKAAEEGAKSTATFEAKFGRASYVGDSSQVED
PGAVGLCEFLKGVQSAL

The amino acid sequence of GCY1-L1-DAK2 is shown, below (SEQ ID NO: 8) with the linker in bold and italic:

MPATLHDSTKILSLNTGAQIPQIGLGTWQSKENDAYKAVLTALKDGYRHI
DTAAIYRNEDQVGQAIKDSGVPREEIFVTTKLWCTQHHEPEVALDQSLKR
LGLDYVDLYLMHWPARLDPAYIKNEDILSVPTKKDGSRAVDITNWNFIKT
WELMQELPKTGKTKAVGVSNFSINNLKDLLASQGNKLTPAANQVEIHPLL
PQDELINFCKSKGIVVEAYSPLGSTDAPLLKEPVILEIAKKNNVQPGHVV
ISWHVQRGYVVLPKSVNPDRIKTNRKIFTLSTEDFEAINNISKEKGEKRV
VHPNWSPFEVFK [symbol] MSAKSFEVTDPVNSSLKGFALANPSITLVPEE
KILFRKTDSDKIALISGGGSGHEPTHAGFIGKGMLSGAVVGEIFASPSTK
QILNAIRLVNENASGVLLIVKNYTGDVLHFGLSAERARALGINCRVAVIG
DDVAVGREKGGMVGRRALAGTVLVHKIVGAFAEEYSSKYGLDGTAKVAKI
INDNLVTIGSSLDHCKVPGRKFESELNEKQMELGMGIHNEPGVKVLDPIP
STEDLISKYMLPKLLDPNDKDRAFVKFDEDDEVVLLVNNLGGVSNFVISS
ITSKTTDFLKENYNITPVQTIAGTLMTSFNGNGFSITLLNATKATKALQS
DFEEIKSVLDLLNAFTNAPGWPIADFEKTSAPSVNDDLLHNEVTAKAVGT
YDFDKFAEWMKSGAEQVIKSEPHITELDNQVGDGDCGYTLVAGVKGITEN
LDKLSKDSLSQAVAQISDFIEGSMGGTSGGLYSILLSGFSHGLIQVCKSK
DEPVTKEIVAKSLGIALDTLYKYTKARKGSSTMIDALEPFVKEFTASKDF
NKAVKAAEEGAKSTATFEAKFGRASYVGDSSQVEDPGAVGLCEFLKGVQS
AL

As described, above, GCY1 and DAK1 enzymes are well-known enzymes and a large number have been cloned and characterized. Accordingly, other GCY1 and DAK1 polypeptides are expected to be suitable for making bifunctional fusion polypeptides, including those described, above. It is also expected that the orientation of the GCY1 and DAK1 polypeptides within the fusion polypeptide is not critical, and that either the GCY1 or the DAK1 polypeptide can be at the N-terminus or C-terminus of the fusion polypeptide.

It is further expected that the GCY1-DAK1 fusion polypeptide can include additional functionalities and structural features, including but not limited to, fluorescent proteins, additional enzymes, antibody tags, antibiotic resistance markers, and the like, which may be at the N-terminus and/or C-terminus of the GCY1-DAK1 fusion polypeptide, may separate the GCY1-DAK1 fusion polypeptide, or may be contained within a linker region (e.g. as described, below) of a GCY1-DAK1 fusion polypeptide.

Various linkers are expected to work as described, including those having at least 70%, at least 80%, at least 85%, at least 90%, at least 95%, or more amino acid sequence identity, to Linker 1 and Linker 2. Future experimentation is likely to identify linkers that optimize enzyme activity. Preferred linkers are peptides but larger linkers, including functional proteins can also be used as linkers. Such linkers may, for example, allow for isolation of an enzyme, provide a fluorescent tag, include an additional enzymatic activity, and the like. Ideally, the linker, GCY1 and DAK1 are a single contiguous amino acid sequence that can be made in a cell using normal translation machinery; however, the principle of linking GCY1 and DAK1 enzymes includes the possibility of a synthetic linker added to GCY1 and DAK1 polypeptides chemically.

In some embodiments, the yeast expressing the bifunctional GCY1-DAK1 fusion polypeptide produce at least 0.5%, at least 0.8%, at least 1%, at least 2%, at least 3%, at least 4%, at least 5%, or even at least 6% more ethanol from a substrate than yeast lacking the bifunctional protein. In some embodiments, the yeast expressing the bifunctional GCY1-DAK1 fusion polypeptide produce at least 0.2%, at least 0.5%, at least 1%, at least 2%, at least 3%, and even at least 4%, more ethanol from a substrate than yeast expressing separate GCY1 and DAK1 polypeptides. In some embodiments, the yeast containing the bifunctional GCY1-DAK1 fusion polypeptide produce at least 3%, at least 5%, at least 10%, at least 15%, or even at least 20%, less glycerol from a substrate than yeast lacking the bifunctional protein. In some embodiments, the yeast containing the bifunctional GCY1-DAK1 fusion polypeptide produce at least 1%, at least 3%, at least 5%, at least 10%, at least 15%, or even at least 20%, less glycerol from a substrate than yeast expressing separate GCY1 and DAK1 polypeptides.

In some embodiments, the present yeast overexpress the GCY1-DAK1 bifunctional polypeptides by at least 50%, at least 100%, at least 200%, at least 300%, at least 500%, or more, and/or at least 2-fold, at least 5-fold, at least 10-fold, at least 20-fold, or more compared to the normal individual expression level of either (or, if specified, both) GCY1 or DAK1, based on mRNA levels.

In some embodiments, the yeast expressing the bifunctional GCY1-DAK1 fusion polypeptide do not additionally overexpress separate GCY1 and DAK1 polypeptides. In other embodiments, the yeast expressing the bifunctional GCY1-DAK1 fusion polypeptide also overexpress separate GCY1 and/or DAK1 polypeptides.

### V. Yeast Additionally Having a Heterologous Phosphoketolase Pathway

Engineered yeast having a heterologous phosphoketolase pathway have been previously described (e.g., WO2015148272, Miasnikov et al.). These cells express heterologous phosphoketolase (PKL) and phosphotransacetylase (PTA), optionally with other enzymes, such as an acetylating acetyl dehydrogenase (AADH) and/or acetyl-CoA synthase (ACS), to channel carbon flux away from the glycerol pathway and toward the synthesis of acetyl-CoA, which is then converted to ethanol.

Such modified cells are capable of increased ethanol production in a fermentation process when compared to otherwise-identical parent yeast cells. The expression of GCY1 and DAK1 can be combined with expression of genes in the PKL pathway to further increase the production of ethanol.

### VI. Combination of GCY1 and DAK1 Expression with Other Genetic Modifications that Benefit Alcohol Production

In some embodiments, in addition to expressing GCY1 and DAK1 as separate polypeptides or as bifunctional fusion polypeptides, the present modified yeast cells include additional modifications that affect ethanol production.

The modified cells may further include mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to acetyl-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lack a heterologous gene(s) encoding an acetylating acetaldehyde dehydrogenase, a pyruvate-formate lyase or both.

In some embodiments, the present modified yeast cells may further overexpress a sugar transporter-like (STL1) polypeptide to increase the uptake of glycerol (see, e.g., Ferreira et al. (2005) Mol Biol Cell 16:2068-76; Dušková et al. (2015) Mol Microbiol 97:541-59 and WO 2015023989 A1). In some embodiments, the present modified yeast cells may further overexpress an ATP-dependent glucose-specific transporter polypeptide to increase the ethanol production.

In some embodiments, the present modified yeast cells further include a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

### VII. Combination of GCY1 and DAK1 Expression with Other Beneficial Mutations

In some embodiments, in addition to expressing GCY1 and DAK1 as separate polypeptides or as bifunctional fusion polypeptides, optionally in combination with other genetic modifications that benefit alcohol production, the present modified yeast cells further include any number of additional genes of interest encoding proteins of interest. Additional genes of interest may be introduced before, during, or after genetic manipulations that result in expression of the fusion polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VIII. Use of the Modified Yeast for Increased Alcohol Production

The present compositions and methods include methods for increasing alcohol production using the modified yeast in fermentation reactions. Such methods are not limited to a particular fermentation process. The present engineered yeast is expected to be a “drop-in” replacement for convention yeast in any alcohol fermentation facility. While primarily intended for fuel ethanol production, the present yeast can also be used for the production of potable alcohol, including wine and beer.

### IX. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase, α-amylase, protease or other enzymes.

### X. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

These and other aspects and embodiments of the present strains and methods will be apparent to the skilled person in view of the present description. The following examples are intended to further illustrate, but not limit, the strains and methods.

### EXAMPLES

### Example 1

### Materials and Methods

### Liquefact Preparation

Liquefact (ground corn slurry) was prepared by adding 600 ppm of urea, 0.124 SAPU/g ds FERMGEN™ 2.5× (acid fungal protease), 0.33 GAU/g ds TrGA variant glucoamylase and 1.46 SSCU/g ds GC626 (Aspergillus α-amylase), adjusted to a pH of 4.8.

### AnKom Assays

300 μL of concentrated yeast overnight culture was added to each of a number ANKOM bottles filled with 30 g prepared liquefact for a final OD of 0.3. The bottles were then incubated at 32° C. with shaking (150 RPM) for 65 hours.

### HPLC Analysis

Samples from serum vial and AnKom experiments were collected in Eppendorf tubes by centrifugation for 12 minutes at 14,000 RPM. The supernatants were filtered with 0.2 μM PTFE filters and then used for HPLC (Agilent Technologies 1200 series) analysis with the following conditions: Bio-Rad Aminex HPX-87H columns, running temperature of 55° C. 0.6 ml/min isocratic flow 0.01 N H₂SO₄, 2.5 μL injection volume. Calibration standards were used for quantification of the of acetate, ethanol, glycerol, and glucose. Samples from shake flasks experiments were collected in Eppendorf tubes by centrifugation for 15 minutes at 14,000 RPM. The supernatants were diluted by a factor of 11 using 5 mM H₂SO₄ and incubated for 5 min at 95° C. Following cooling, samples were filtered with 0.2 μM Corning FiltrEX CLS3505 filters and then used for HPLC analysis. 10 μl was injected into an Agilent 1200 series HPLC equipped with a refractive index detector. The separation column used was a Phenomenex Rezex-RFQ Fast Acid H+ (8%) column. The mobile phase was 5 mM H₂SO₄, and the flow rate was 1.0 mL/min at 85° C. HPLC Calibration Standard Mix from Bion Analytical was used for quantification of the of acetate, ethanol, glycerol, and glucose. Unless otherwise specified, all values are expressed in g/L.

### Growth Determination

Two 96-well microtiter plates (MTP) containing 500 μL of YPD were inoculated with 20 μL concentrated yeast overnight culture to final OD of 0.1. The MTPs were incubated at 32° C. or 36° C. with shaking (150 RPM) for 24 hours. The yeast cultures were diluted 50 times in demineralized water to a final volume of 100 μL in 500 μL volume flat bottom transparent plates (Greiner Bio-one 655101). The OD was measured at a wavelength of 660 nm.

### Example 2

### Construction of Plasmids Harboring Fusion Genes Encoding an N-Terminal Glycerol Dehydrogenase and a C-Terminal Dihydroxyacetone Kinase

Synthetic GCY1-L1-DAK1 and GCY1-L2-DAK1 are fusion genes that include codon optimized glycerol dehydrogenase (GCY1, SEQ ID NO: 2) and dihydroxyacetone kinase (DAK1, SEQ ID NO: 1) fused by linker 1 (SEQ ID NO: 3) and linker 2 (SEQ ID NO: 4), respectively. The amino acid sequence of the fusion polypeptides GCY-L1-DAK1, and GCY1-L2-DAK1 are represented by SEQ ID NOs: 5 and 6, respectively.

Plasmid pZKIIC-YL1K contains the expression cassette to express the GCY1-L1-DAK1 fusion polypeptide under the control of an ACT1 promoter (YFL039C locus) and FBA1 terminator (YKL060C locus). Plasmid pZKIIC-YL1K was designed to integrate the expression cassette into the positions of 345856 and 350891 of Saccharomyces chromosome II. The functional and structural composition of plasmid pZKIIC-YL1K is described in Table 2.

TABLE 2
Functional and structural elements of plasmid pZKIIC-YL1K

| Sequence | Functional/Structural | |
|---|---|---|
| positions | element | Description |
| 316-716 | “IIC-Down” fragment, | 401-bp DNA fragment from |
| | between positions of | S. cerevisiae |
| | 350491 and 350891 of | |
| | chromosome II, strain | |
| | S288c | |
| 717-3112 | ColE1 replicon and | These sequences are not part of |
| | ampicillin resistance | the DNA fragment integrated into |
| | marker gene | yeast genome |
| 3113-3543 | “IIC-Up” fragment, | 431-bp DNA fragment from |
| | between positions of | S. cerevisiae |
| | 349856 and 350285 of | |
| | chromosome II, strain | |
| | S288c | |
| 3611-303 | ACT1 Promoter:: | Cassette for expression of codon |
| | GCY1-L1-DAK1:: | optimized GCY1-L1-DAK1 |
| | FBA1 Terminator | encoding both glycerol |
| | | dehydrogenase and dihydroxy- |
| | | acetone kinase, derived from |
| | | S. cerevisiae |

Plasmid pZKIIC-YL2K contains the expression cassette to express the GCY1-L2-DAK1 fusion polypeptide under the control of an ACT1 promoter (YFL039C locus) and FBA1 terminator (YKL060C locus). Plasmid pZKIIC-YL1K was designed to integrate the expression cassette into the positions of 345856 and 350891 of Saccharomyces chromosome II. The functional and structural composition of plasmid pZKIIC-YL2K is exactly the same as described in Table 2, except that the expression cassette is ACT1Promoter::GCY1-L2-DAK1::FBA1Terminator.

### Example 3

### Construction of Plasmids Harboring Fusion Genes Encoding an N-Terminal Dihydroxyacetone Kinase and a C-Terminal Glycerol Dehydrogenase

Synthetic DAK1-L1-GCY1 and DAK1-L2-GCY1 are fusion genes that include codon optimized dihydroxyacetone kinase (DAK1, SEQ ID NO: 1) and glycerol dehydrogenase (GCY1, SEQ ID NO: 2) fused by linker 1 (SEQ ID NO: 3) and linker 2 (SEQ ID NO: 4), respectively. The amino acid sequence of the fusion polypeptides DAK-L1-GCY1 and DAK1-L2-GCY1 are represented by SEQ ID NOs: 7 and 8, respectively.

Plasmids pZKIIC-KL1Y and pZKIIC-KL2Y contain the expression cassette to express the DAK-L1-GCY1 and DAK1-L2-GCY1 fusion polypeptide under the control of an ACT1 promoter (YFL039C locus) and FBA1 terminator (YKL060C locus). Both pZKIIC-KL1Y and pZKIIC-KL2Y were designed to integrate the expression cassette into the positions of 345856 and 350891 of Saccharomyces chromosome II. The functional and structural composition of plasmids pZKIIC-KL1Y and pZKIIC-KL2Y were exactly the same as described in Table 2, except that the expression cassette was ACT1Promoter::DAK1-L1-GCY1::FBA1Terminator in plasmid pZKIIC-KL1Y and ACT1Promoter::DAK1-L2-GCY1::FBA1Terminator in pZKIIC-KL2Y.

### Example 4

### Plasmid pZKIIC-HYWK with GCY1 and DAK1 as Individual Genes

Plasmid pZKIIC-HYWK was designed as a control for testing the effect of plasmids for the expression of the GCY1-DAK1 bifunctional fusion proteins described, above. pZKIIC-HYWK contains two express cassettes. The expression of the codon optimized GCY1 and DAK1 are individually controlled. The expression of GCY1 is under the control of HXT3 promoter and FBA1 terminator, while the expression of the codon optimized DAK1 is under the control of CWP2 promoter (YKL096W-A locus) and PGK1 terminator (YCR012W locus). RNAseq data (see, e.g., Wang, Z. et al. (2009) Nature Rev. Gen. 10:57-63) suggests that the transcription activities of HXT3 and CWP2 promoters are much stronger than the ACT1 promoter used for expression of GCY1 and DAK1 fusion genes. Plasmid pZKIIC-HYWK was designed to integrate the two expression cassettes into the positions of 345856 and 350891 of Saccharomyces chromosome II. The functional and structural composition of plasmid pZKIIC-HYWK is described in Table 3.

TABLE 3
Functional and structural elements of plasmid pZKIIC-HYWK

| Sequence | Functional/Structural | |
|---|---|---|
| positions | element | Description |
| 2-402 | “IIC-Down” fragment, | 401-bp DNA fragment from |
| | between positions of | S. cerevisiae |
| | 350491 and 350891 of | |
| | chromosome II, strain | |
| | S288c | |
| 403-2798 | ColE1 replicon and | These sequences are not part of |
| | ampicillin resistance | the DNA fragment integrated into |
| | marker gene | yeast genome |
| 2799-3229 | “IIC-Up” fragment, | 431-bp DNA fragment from |
| | between positions of | S. cerevisiae |
| | 349856 and 350285 of | |
| | chromosome II, strain | |
| | S288c | |
| 3302-6098 | CWP2 Promoter:: | Cassette for expression of codon |
| | DAK1:: | optimized DAK1 encoding |
| | PGK1 Terminator | dihydroxyacetone kinase, |
| | | derived from S. cerevisiae |
| 6114-8051 | HXT3 Promoter:: | Cassette for expression of codon |
| | GCY1:: | optimized GCY1 encoding |
| | FBA1 Terminator | glycerol dehydrogenase, derived |
| | | from S. cerevisiae |

### Example 5

### Generation an FG-Ura3 Strain with a Ura3 Genotype

The S. cerevisiae strain, FERMAX′ Gold Label (Martrex Inc., Minnesota, USA; abbreviated, “FG”) is well-known in the grain ethanol industry and was used as the parental, “wild-type” strain to make the present engineered yeast.

Plasmid pTOPO II-Blunt ura3-loxP-KanMX-loxP-ura3 was designed to replace the URA3 gene in strain FG with mutated ura3 and URA3-loxP-TEFp-KanMX-TEFt-loxP-URA3 fragments. The functional and structural elements of the plasmid are listed in Table 4.

TABLE 4
Functional/structural elements of pTOPO II-Blunt
ura3-loxP-KanMX-loxP-ura3

| Sequence Positions | Functional/Structural | |
|---|---|---|
| (bp) | Element | Comment |
| 763-1695 | KanR gene in E. coli | Vector sequence |
| 2388-3061 | pUC origin | Vector sequence |
| 3520-3569, | URA3 3′-flanking | Synthetic DNA identical |
| reverse | region, | to S. cerevisiae genomic |
| orientation | | sequence to URA3 locus |
| 3601-3634, | loxP66 | Synthetic DNA identical |
| reverse | | to loxP66 consensus |
| orientation | | |
| 3635-5400, | TEF1::KanMX4::TEF | KanMX expression |
| reverse | Terminator | cassette |
| orientation | | |
| 5406-5439, | loxP71 | Synthetic DNA identical |
| reverse | | to loxP71 consensus |
| orientation | | |
| 5470-5519, | URA3 5′-flanking | Synthetic DNA identical |
| reverse | region | to the URA3 locus on |
| orientation | | the S. cerevisiae genome |

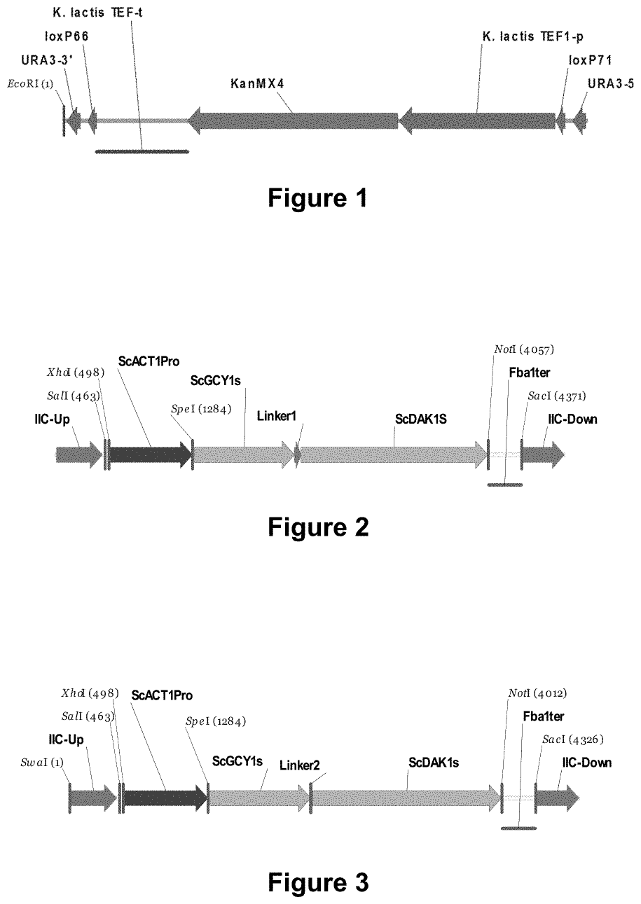

A 2,018-bp DNA fragment containing the ura3-loxP-KanMX-loxP-ura3 cassette was released from plasmid TOPO II-Blunt ura3-loxP-KanMX-loxP-ura3 by EcoRI digestion (FIG. 1). The fragment was used to transform S. cerevisiae FG cells by electroporation. Transformed colonies able to grow on media containing G418 were streaked on synthetic minimal plates containing 20 μg/ml uracil and 2 mg/ml 5-fluoroorotic acid (5-FOA). Colonies able to grow on 5-FOA plates were further confirmed for URA3 deletion by growth of phenotype on SD-Ura plates, and by PCR. The ura3 deletion transformants were unable to grow on SD-Ura plates. A single 1.98-kb PCR fragment was obtained with test primers. In contrast, the same primer pairs generated a 1.3-kb fragment using DNA from the parental FG strain, indicating the presence of the intact ura3 gene. The ura3 deletion strain was named as FG-KanMX-ura3.

To remove the KanMX expression cassette from strain FG-KanMX-ura3, plasmid pGAL-Cre-316 was used to transform cells of strain FG-KanMX-ura3 by electroporation. The purpose of using this plasmid is to temporary express the Cre enzyme, so that the LoxP-sandwiched KanMX gene will be removed from strain FG-KanMX-ura3 to generate strain

FG-ura3. pGAL-Cre-316 is a self-replicating circular plasmid that was subsequently removed from strain FG-ura3. None of the sequence elements from pGAL-cre-316 were inserted into the strain FG-ura3 genome. The functional and structural elements of plasmid pGAL-Cre-316 is listed in Table 5.

TABLE 5
Functional and structural elements of pGAL-Cre-316.

| Sequence positions (bp) | Functional/Structural element |
|---|---|
| 1-4810 | Yeast-bacterial shuttle vector pRS316 |
| | sequence |
| 440-1059 | pBR322 origin of replication |
| 2984-4080 | S. cerevisiae URA3 gene |
| 4355-4454 | F1 origin |
| 4813-6603, reverse orientation | GALp-Cre-ADHt cassette, reverse |
| | orientation |

The transformed cells were plated on SD-Ura plates. Single colonies were transferred onto a YPG plate and incubated for 2 to 3 days at 30° C. Colonies were then transferred to new a YPD plate for 2 additional days. Finally, cell suspensions from the YPD plate were spotted on to following plates: YPD, G418 (150 μg/ml), 5-FOA (2 mg/ml) and SD-Ura. Cells able to grow on YPD and 5-FOA, and unable to grow on G418 and SD-Ura plates, were picked for PCR confirmation as described, above. The expected PCR product size was 0.4-kb and confirmed the identity of the KanMX (geneticin)-sensitive, ura3-deletion strain, derived from FG-KanMX-ura3. This strain was named as FG-ura3.

### Example 7

### Generation of Strains Expressing GCY1 and DAK1 as a Fusion Polypeptide or as Separate Polypeptides

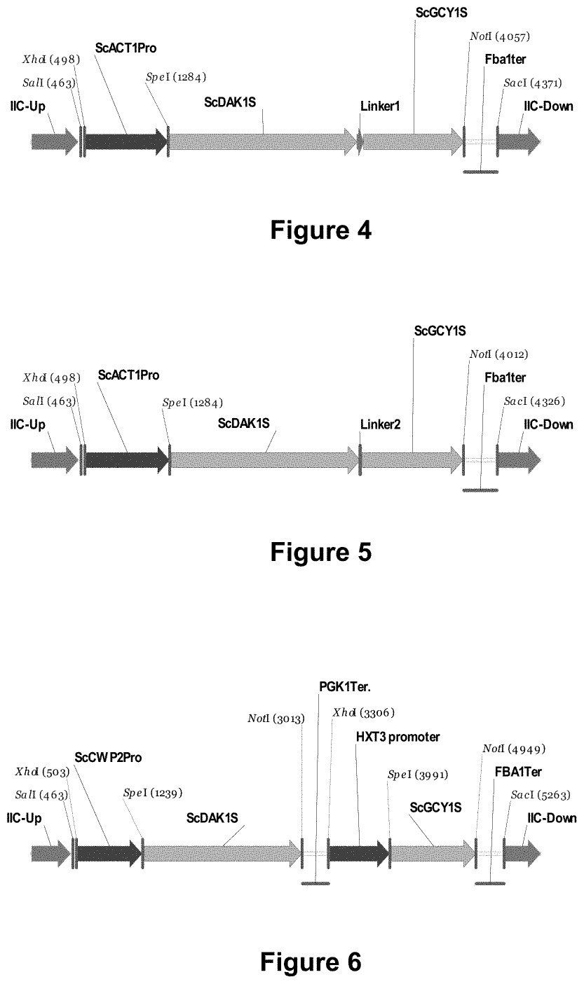

The FG-ura3 strain was used as parental yeast for the introduction of the GCY1 and DAK1 genes described, above. Cells were transformed with either (i) a 4,776-bp SwaI fragment containing the GCY1-L1-DAK1 expression cassette from plasmid pZKIIC-YL1K (FIG. 2), (ii) a 4,731-bp SwaI fragment containing GCY1-L2-DAK1 expression cassette from plasmid pZKIIC-YL2K (FIG. 3), (iii) a 4776-bp SwaI fragment containing the DAK1-L1-GCY1 expression cassette from plasmid pZKIIC-KL1Y (FIG. 4), (iv) a 4,731-bp SwaI fragment containing DAK1-L2-GCY1 expression cassette from plasmid pZKIIC-KL2Y (FIG. 5), or (v) a 5,668-bp SwaI fragment containing GCY1 and DAK1 individual expression cassettes from plasmid pZKIIC-HYKK (FIG. 6). Transformants were selected and designated as shown in Table 6.

TABLE 6
Designation of selected transformants

| | | | Transgene(s) |
|---|---|---|---|
| Strain | Insert | Integration locus | expressed |
| G1424 | SwaI fragment | Positions of 350491 and | DAK1-L1-GCY1 |
| | from | 350891 of chromosome | fusion protein |
| | pZKIIC-KL1Y | II, strain S288c | |
| G1426 | SwaI fragment | Positions of 350491 and | DAK1-L2-GCY1 |
| | from | 350891 of chromosome | fusion protein |
| | pZKIIC-KL2Y | II, strain S288c | |
| G1429 | SwaI fragment | Positions of 350491 and | GCY1-L1-DAK1 |
| | from | 350891 of chromosome | fusion protein |
| | pZKIIC-YL1K | II, strain S288c | |
| G1430 | SwaI fragment | Positions of 350491 and | GCY1-L2-DAK1 |
| | from | 350891 of chromosome | fusion protein |
| | pZKIIC-YL2K | II, strain S288c | |
| G1537 | SwaI fragment | Positions of 350491 and | GCY1 and DAK1 |
| | from | 350891 of chromosome | individual |
| | pZKIIC-HYWK | II, strain S288c | |

### Example 8

### Comparison of Strains Expressing GCY1 and DAK1 as a Fusion Polypeptide or as Separate Polypeptides in AnKom Assays

To determine the benefits of the bifunctional proteins, the performance of strains G1424, G1426, G1429, G1430 and G1537 were analyzed in AnKom assays, as described in Example 1. Performance in terms of ethanol, glycerol and acetate production (in g/L) is shown in Table 7.

TABLE 7
FG versus G1424, G1426, G1429 and G1430 in AnKom assays

| Strain | Transgene(s) expressed | EtOH | Glycerol | Acetate |
|---|---|---|---|---|
| FG | None | 143.14 | 15.96 | 0.56 |
| G1424 | DAK1-L1-GCY1 fusion protein | 144.00 | 15.92 | 0.75 |
| G1426 | DAK1-L2-GCY1 fusion protein | 143.82 | 15.91 | 0.72 |
| G1429 | GCY1-L1-DAK1 fusion protein | 144.33 | 15.78 | 0.71 |
| G1430 | GCY1-L2-DAK1 fusion protein | 144.00 | 15.90 | 0.63 |

TABLE 8
FG versus G1537 in AnKom assays

| Strain | Transgene(s) expressed | EtOH | Glycerol | Acetate |
|---|---|---|---|---|
| FG | None | 143.10 | 16.23 | 0.91 |
| G1537 | DAK1 and GCY1, | 143.98 | 16.53 | 0.98 |
| | individual | | | |

Although the GCY1-DAK1 fusion genes were under the control of the ACT1 promoter, which is much weaker than the HXT3 and CWP2 promoters used for individual expression of GCY1 and DAK1 in strain G1537, the increase in ethanol production with the G1424, G1426, G1429 and G1430 strains was 0.6, 0.5, 0.8 and 0.6%, respectively, compared to 0.6% with the G1537 strain.

### Example 9

### Construction of a Plasmid Encoding a PKL-PTA Bifunctional Fusion Protein

Synthetic phosphoketolase and phosphotransacetylase fusion gene 1, GvPKL-L1-LpPTA, includes the codon-optimized coding regions for the phosphoketolase from Gardnerella vaginalis (GvPKL) and the phosphotransacetylase from Lactobacillus plantarum (LpPTA) joined with synthetic linker L1 (SEQ ID NO: 3) and the short amino acid sequence VTS, which provides an extra nine nucleotides for addition of a restriction enzyme site to facilitate the cloning. The amino acid sequence of the resulting fusion polypeptide is represented by SEQ ID NO: 9.

Amino acid sequence of GvPKL-L1-LpPTA is shown, below (SEQ ID NO: 9), with the linker and VTS in bold and italic:

MTSPVIGTPWKKLNAPVSEAAIEGVDKYWRVANYLSIGQIYLRSNPLMKE
PFTREDVKHRLVGHWGTTPGLNFLIGHINRFIAEHQQNTVIIMGPGHGGP
AGTAQSYLDGTYTEYYPKITKDEAGLQKFFRQFSYPGGIPSHFAPETPGS
IHEGGELGYALSHAYGAVMNNPSLFVPAIVGDGEAETGPLATGWQSNKLV
NPRTDGIVLPILHLNGYKIANPTILSRISDEELHEFFHGMGYEPYEFVAG
FDDEDHMSIHRRFADMFETIFDEICDIKAEAQTNDVTRPFYPMIIFRTPK
GWTCPKFIDGKKTEGSWRAHQVPLASARDTEAHFEVLKNWLKSYKPEELF
NEDGSIKEDVLSFMPQGELRIGQNPNANGGRIREDLKLPNLDDYEVKEVK
EFGHGWGQLEATRRLGVYTRDVIKNNPDSFRIFGPDETASNRLQAAYEVT
NKQWDAGYLSELVDEHMAVTGQVTEQLSEHQMEGFLEAYLLTGRHGIWSS
YESFVHVIDSMLNQHAKWLEATVREIPWRKPISSMNLLVSSHVWRQDHNG
FSHQDPGVTSVLLNKTFNNDHVIGIYFPVDSNMLLAVGEKVYKSTNMINA
IFAGKQPAATWLTLDEAREELEKGAAEWKWASNAKNNDEVQVVLAGIGDV
PQQELMAAADKLNKLGVKFKVVNIVDLLKLQSAKENNEALTDEEFTELFT
ADKPVLLAYHSYAHDVRGLIFDRPNHDNFNVHGYKEQGSTTTPYDMVRVN
DMDRYELTAEALRMVDADKYADEIKKLEDFRLEAFQFAVDKGYDHPDYTD
WVWPGVKTDKPGAVTATAATAGDNE [symbol] MD
LFESLAQKITGKDQTIVFPEGTEPRIVGAAARLAADGLVKPIVLGATDKV
QAVANDLNADLTGVQVLDPATYPAEDKQAMLDALVERRKGKNTPEQAAKM
LEDENYFGTMLVYMGKADGMVSGAIHPTGDTVRPALQIIKTKPGSHRISG
AFIMQKGEERYVFADCAINIDPDADTLAEIATQSAATAKVFDIDPKVAML
SFSTKGSAKGEMVTKVQEATAKAQAAEPELAIDGELQFDAAFVEKVGLQK
APGSKVAGHANVFVFPELQSGNIGYKIAQRFGHFEAVGPVLQGLNKPVSD
LSRGCSEEDVYKVAIITAAQGLA

Plasmid pZK41W-GLAF12 contains three cassettes to express the GvPKL-L1-LpPTA fusion polypeptide, acylating acetaldehyde dehydrogenase from Desulfospira joergensenii (DjAADH), and acetyl-CoA synthase from Methanosaeta concilii (McACS). Both DjAADH and McACS were codon optimized. The expression of GvPKL-L1-LpPTA is under the control of an HXT3 promoter and FBA1 terminator. The expression of DjAADH is under the control of TDH3 promoter and ENO2 terminator. The expression of McACS is under the control of PDC/promoter and PDC1 terminator. Plasmid pZK41W-GLAF12 was designed to integrate the three expression cassettes into the Saccharomyces chromosome downstream of the YHL041W locus. The functional and structural composition of plasmid pZK41W-GLAF12 is described in Table 9.

TABLE 9
Functional and structural elements of plasmid pZK41W-GLAF12

| Location | Functional/Structural | |
|---|---|---|
| (bp) | element | Description |
| 4-81 | “Down” fragment, | 78-bp DNA fragment from |
| | downstream of | S. cerevisiae |
| | YHL041W locus | |
| 120-153 | LoxP71 site | LoxP71 site |
| 154-1442 | Ura3 gene | Ura3 gene used as selection |
| | | marker |
| 1443-1476 | LoxP66 | LoxP66 site |
| 1483-1562 | “M” fragment, | 80-bp DNA fragment from |
| | downstream of | S. cerevisiae |
| | YHL041W locus | |
| 1563-4242 | ColE1 replicon and | These sequences are not part of |
| | ampicillin resistance | the DNA fragment integrated |
| | marker gene | into yeast genome |
| 4243-4318 | “Up” fragment, | 76-bp DNA fragment from |
| | downstream of | S. cerevisiae |
| | YHL041W locus | |
| 4416-7804 | PDC1Promoter:: | Cassette for expression of codon |
| | McACS:: | optimized McACS encoding |
| | PDC Terminator | acetyl-CoA synthase, |
| | | derived from M. consilii |
| 7873-10463 | TDH3 Promoter:: | Cassette for expression of codon |
| | DjAADH:: | optimized DjAADH encoding |
| | ENO Terminator | acylating acetaldehyde |
| | | dehydrogenase, derived from |
| | | D. joergensenii |
| 10497-15037 | HXT3 Promoter:: | Cassette for expression of codon- |
| | GvPKL-L1-LpPTA:: | optimizedGvPKL-L1-LpPTA |
| | FBA1 Terminator. | fusion gene encoding |

### Example 10

### Generation of Strain Expressing PKL and PTA as a Fusion Polypeptide

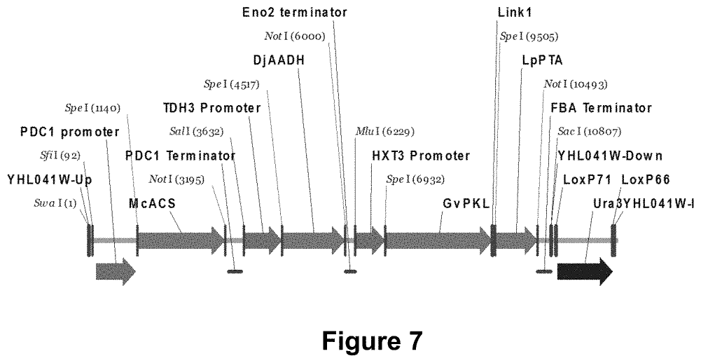

The FG-ura3 strain (Example 5) was used as a parent to introduce the GvPKL-L1-LpPTA genes described, above. Cells were transformed with a 12,372-bp SwaI fragment of pZK41W-GLAF12 containing the expression cassettes for PKL pathway (FIG. 7). Transformant was selected and designated as shown in Table 10.

TABLE 10
Designation of selected transformant

| | | Integration | |
|---|---|---|---|
| Strain | Insert | locus | Transgene(s) expressed |
| G176 | SwaI fragment from | YHL041W | GvPKL-L1-LpPTA fusion |
| | pZK41W-GLAF12 | | protein, DjAADH and |
| | | | McACS |

### Example 11

### Analysis of Yeast Expressing a PKL-PTA Bifunctional Fusion Polypeptide

To determine the benefit of the bifunctional GvPKL-L1-LpPTA polypeptide, the performances of strain G176 and its parent strain FG were precisely analyzed in AnKom assays, as described in Example 1. Performance in terms of ethanol, glycerol and acetate production (in g/L) is shown in Table 11.

TABLE 11
FG versus G176 in AnKom assays

| Strain | Transgene(s) expressed | EtOH | Glycerol | Acetate |
|---|---|---|---|---|
| FG | none | 134.26 | 17.24 | 0.78 |
| G176 | GvPKL-L1-LpPTA fusion | 142.02 | 14.73 | 1.26 |
| | protein, DjAADH, and McACS | | | |

The increase in ethanol production with the G176 strain was about 5.8% over its parent FG strain.

### Example 12

### Generation of Strains Expressing GvPKL-L1-LpPTA Together with GCY1 and DAK1 as a Fusion Polypeptide or as Separate Polypeptides

Yeast strain expressing GvPKL-L1-LpPTA fusion protein together the fused or non-fused GCY1 and DAK1 genes, were constructed by transforming strain with GvPKL-L1-LpPTA fusion with either (i) a 4,776-bp SwaI fragment containing the GCY1-L1-DAK1 expression cassette from plasmid pZKIIC-YL1K (FIG. 2), (ii) a 4,731-bp SwaI fragment containing GCY1-L2-DAK1 expression cassette from plasmid pZKIIC-YL2K (FIG. 3), (iii) a 4776-bp SwaI fragment containing the DAK1-L1-GCY1 expression cassette from plasmid pZKIIC-KL1Y (FIG. 4), (iv) a 4,731-bp SwaI fragment containing DAK1-L2-GCY1 expression cassette from plasmid pZKIIC-KL2Y (FIG. 5), or (v) a 5,668-bp SwaI fragment containing GCY1 and DAK1 individual expression cassettes from plasmid pZKIIC-HYKK (FIG. 6). Transformants were selected and designated as shown in Table 12. All strains express the GvPKL-L1-LpPTA fusion protein.

TABLE 12
Designation of selected transformant

| Strain | GCY1 and DAK1 transgene(s) expressed |
|---|---|
| G1272 | GCY1-L1-DAK1 fusion protein |
| G1275 | GCY1-L2-DAK1 fusion protein |
| G1277 | DAK1-L1-GCY1 fusion protein |
| G1280 | DAK1-L2-GCY1 fusion protein |
| G1532 | GCY1 and DAK1, individual |

### Example 9

### Comparison of Strains Expressing GvPKL-L1-LpPTA Together with GCY1 and DAK1 as a Fusion Polypeptide or as Separate Polypeptides in AnKom Assays

To determine the benefits of the GCY1-DAK1 bifunctional proteins in the strain G176 expressing GvPKL-L1-LpPTA, DjAADH, and McACS, the performance of strains G1272, G1275, G1277, G1280 and G1532 were analyzed in AnKom assays, as described in Example 1. Performance in terms of ethanol, glycerol and acetate production (in g/L) is shown in Tables 13 and 14.

TABLE 13
G176 versus G1272, G1275, G1277, G1280 in AnKom assays

| | GCY1 and DAK1 Transgene(s) | | | |
|---|---|---|---|---|
| Strain | expressed | EtOH | Glycerol | Acetate |
| G176 | none | 144.85 | 11.22 | 1.42 |
| G1272 | GCY1-L1-DAK1 fusion | 146.72 | 11.49 | 1.44 |
| G1275 | GCY1-L2-DAK1 fusion | 147.33 | 11.54 | 1.42 |
| G1277 | DAK1-L1-GCY1 fusion | 146.63 | 11.54 | 1.42 |
| G1280 | DAK1-L2-GCY1 fusion | 147.02 | 11.64 | 1.45 |

Although the GCY1-DAK1 fusion genes were under the control of the ACT1 promoter, which is much weaker than the HXT3 and CWP2 promoters used for individual expression of GCY1 and DAK1 in strain G1532 (see below), the increase in ethanol production with the G1272, G1275, G1277 and G1280 strains was about 1.3, 1.7, 1.2 and 1.5%, respectively over its parent strain G176. These observed differences are approximately twice those as observed in Example 8, in which the parental yeast was the FG strain, suggesting that the GCY1-DAK1 bifunctional fusion polypeptides confers a greater benefit in engineered yeast that additionally harbor an exogenous phosphoketolase pathway.

TABLE 14
G176 versus G1532 in AnKom assays

| | GCY1 and DAK1 Transgene(s) | | | |
|---|---|---|---|---|
| Strain | expressed | EtOH | Glycerol | Acetate |
| G176 | none | 142.90 | 15.53 | 1.61 |
| G1532 | GCY1 and DAK1, individual | 144.10 | 15.19 | 1.53 |

The increase in ethanol production with the G1532 strain was about 0.8% over its parent strain G176, suggesting that overexpression of GCY1 and DAK1 protein, separately, increases ethanol production in engineered yeast that additionally harbor an exogenous phosphoketolase pathway. However, expression of the GCY1-DAK1 bifunctional fusion polypeptides confers an approximately 2-fold greater benefit in such engineered yeast compared to the overexpression of the GCY1-DAK1 polypeptides, separately.

What is claimed is:

1. A fusion polypeptide comprising a first amino acid sequence having glycerol dehydrogenase activity fused to a second amino acid sequence having dihydroxyacetone kinase activity, wherein the fusion polypeptide, when expressed in a yeast cell, is capable of converting glycerol to dihydroxyacetone phosphate.

2. The fusion polypeptide of claim 1, wherein the first amino acid sequence and second amino acid sequence are fused via a linker peptide.

3. The fusion polypeptide of claim 1 or 2, wherein the first amino acid sequence is present at the N-terminus of the fusion polypeptide and second amino acid sequence is present at the C-terminus of the fusion polypeptide.

4. The fusion polypeptide of claim 1 or 2, wherein the second amino acid sequence is present at the N-terminus of the fusion polypeptide and first amino acid sequence is present at the C-terminus of the fusion polypeptide.

5. The fusion polypeptide of any of the preceding claims, wherein the first amino acid sequence is the glycerol dehydrogenase from a Saccharomyces sp. or a structural or functional homolog, thereof.

6. The fusion polypeptide of any of the preceding claims, wherein the second amino acid sequence is the dihydroxyacetone kinase from a Saccharomyces sp. or a structural or functional homolog, thereof.

7. A DNA sequence encoding the fusion polypeptide of any of the preceding claims, optionally capable of overexpressing the fusion polypeptide compared to the individual expression levels of either or both GCY1 or DAK1, based on mRNA levels, compared to a parental yeast not harboring the DNA sequence.

8. Yeast cells comprising the DNA sequence of claim 7.

9. Yeast cells expressing or overexpressing the fusion polypeptide of any of claims 1-8.

10. The yeast cells of claim 9, wherein the yeast cells further comprise an exogenous phosphoketolase pathway.

11. The yeast cells of any of claim 9 or 10, wherein the yeast cells further comprise an alteration in the glycerol pathway and/or the acetyl-CoA pathway.

12. The yeast cells of any of claims 9-11, wherein the yeast cells do not additionally overexpress separate polypeptides having glycerol dehydrogenase activity and/or dihydroxyacetone kinase activity.

13. Modified yeast cells comprising a genetic modification that causes the cells to overexpress a polypeptide having glycerol dehydrogenase activity and overexpress a polypeptide having dihydroxyacetone kinase activity, and which modified yeast cells further comprise an exogenous phosphoketolase pathway.

14. The modified yeast cells of claim 13, wherein the polypeptide having glycerol dehydrogenase activity and the polypeptide having dihydroxyacetone kinase activity are produced as a bifunctional fusion polypeptide capable of converting glycerol to dihydroxyacetone phosphate.

15. The modified yeast cells of claim 13 or 14, wherein the yeast produce a reduced amount of dihydroxyacetone compared to otherwise unmodified or parental yeast.

16. The modified yeast cells of any of claims 13-15 further comprising an alteration in the glycerol pathway and/or the acetyl-CoA pathway

17. The modified yeast of any of claims 13-16, further comprising an exogenous gene encoding a carbohydrate processing enzyme.

18. The modified yeast cells of any of claims 13-17, wherein the yeast cells are from a Saccharomyces spp.

19. A method for increasing alcohol production by yeast cells during fermentation of a carbohydrate substrate, comprising contacting the carbohydrate substrate with modified yeast cells having an exogenous phosphoketolase pathway and producing a polypeptide having glycerol dehydrogenase activity and a polypeptide having dihydroxyacetone kinase activity, wherein the modified yeast cells produce during fermentation an increased amount of ethanol compared to yeast cells that do not produce the polypeptide having glycerol dehydrogenase activity and/or the polypeptide having dihydroxyacetone kinase activity.

20. The method of claim 19, wherein the modified yeast cells are the yeast cells of any of claims 9-11.

21. The method of any claim 19 or 20, wherein the polypeptide having glycerol dehydrogenase activity and the polypeptide having dihydroxyacetone kinase activity are expressed as a bifunctional fusion polypeptide capable of converting glycerol to dihydroxyacetone phosphate.

22. The method of claim 21, wherein the bifunctional fusion polypeptide is the fusion polypeptide of any of claims 1-6.
